(19) United States
(12) Patent Application Publication
(10) Pub. No.: US 2010/0021538 A1
(43) Pub. Date: Jan. 28, 2010

(54) PHARMACEUTICAL COMPOSITIONS CONTAINING HEPARIN DERIVATIVES

(76) Inventors: Youngro Byun, Seoul (KR); Jinwoo Park, Seoul (KR); Ok-Cheol Jeon, Seoul (KR); Hyun Tae Moon, Seoul (KR)

Correspondence Address: ALAN J. HOWARTH, P.O. BOX 1909, SANDY, UT, 84091-1909, US

(21) Appl. No.: 12/414,549

(22) Filed: Mar. 30, 2009

Related U.S. Application Data
(63) Continuation-in-part of application No. 12/286,535, filed on Sep. 29, 2008, now abandoned.
Application No. 12/286,535 is a continuation-in-part of application No. 12/074,269, filed on Feb. 29, 2008, now abandoned.

(51) Int. Cl. A61K 9/48; A61K 31/727; A61K 9/00; A61K 9/16; A61K 9/28; A61P 7/04

(52) U.S. Cl. 424/463; 514/56; 424/400; 424/474; 424/490

(57) ABSTRACT

An oral pharmaceutical composition for anticoagulation of blood includes a conjugate of an anticoagulation polysaccharide, such as heparin, covalently bonded to a hydrophobic agent wherein the conjugate is mixed with a solubilizer for inhibiting self-aggregation of the conjugate into nanoparticles. The composition can be coated, formed into tablets, or placed in capsules. Methods of making these oral formulations are also described.

### CROSS-REFERENCE TO RELATED APPLICATIONS

This application is a continuation-in-part of U.S. application Ser. No. 12/286,535, filed Sep. 29, 2008, now abandoned, which is a continuation-in-part of U.S. application Ser. No. 12/074,269, filed Feb. 29, 2008, now abandoned, both of which are hereby incorporated by reference in their entireties.

### STATEMENT REGARDING FEDERALLY SPONSORED RESEARCH OR DEVELOPMENT

Not applicable.

### BACKGROUND OF THE INVENTION

This invention relates to pharmaceutical compositions. More particularly, this invention relates to pharmaceutical compositions, illustrative embodiments of which comprise an amphiphilic heparin derivative, a solubilizer, and pharmaceutical excipients; oral formulations prepared from such pharmaceutical compositions; and methods of preparing the pharmaceutical compositions and oral formulations.

Heparin has been widely used as one of the most powerful anticoagulants used to prevent and treat deep vein thrombosis (DVT) or pulmonary embolism (PE) (Damus et al., Heparin-A generalized view of its anticoagulant action, 246 Nature 355-356 (1973); L. Jin et al., The anticoagulant activation of antithrombin by heparin, 94 Proc. Nat'l Acad. Sci. USA 14683-14688 (1997)). However, heparin treatment can presently be given only through injection, thus making it available only for hospitalized patients (R. D. Rosenberg, Biochemistry and Pharmacology of Low Molecular Weight Heparin, 34 Semin. Hematol., 2-8 (1997); G. F. Pineo & R. D. Hull, Unfractionated and Low Molecular-weight Heparin, 82 Curr. Concepts Thromb. 587-599 (1998)). As a result, patients who have been on heparin treatment by parenteral (intravenous or subcutaneous) injection are switched to oral warfarin treatment upon discharge from the hospital. Warfarin has a slow onset and high risk of drug-to-drug interaction. Therefore, there has long been great interest in research for heparin pharmaceutical compositions and methods of oral administration to treat DVT or PE patients.

Heparin reportedly cannot be absorbed in the GI tract owing to its large molecular size and high negative charge density (L. B. Haques, Heparins: Anionic polyelectrolyte drugs, 31 Pharmacology Res. 100-166 (1980)). Heparin difficultly penetrates epithelial cells due to its hydrophilicity, which causes repulsion by polar groups of the epithelial membrane, as well as low penetration rate (D. A. Norris et al., The effect of physical barriers and properties on the oral absorption of particulates, 34 Advanced Drug Delivery Reviews 135-154 (1998)).

When heparin was prepared in an aerosol formulation and combined with lipophilic agents or membrane permeation enhancing agents, heparin was not detected in blood (A. Dalpozzo et al., New heparin complexes active by intestinal absorption. I. Multiple ion pairs with basic organic compounds, 56 Thromb. Res. 119-124 (1989)).

While prior heparin-containing products and methods of use thereof are known and are generally suitable for their limited purposes, they possess certain inherent deficiencies that detract from their overall utility as oral formulations. For example, some formulations self-assemble into nanoparticles under aqueous conditions and thus are transported through the membrane in the GI tract with difficulty. Other formulations are difficult or expensive to make, or contain residual organic solvents that cause toxicity problems.

In view of the foregoing, it will be appreciated that providing an oral formulation of heparin that is easy to make, inexpensive, and nontoxic would be a significant advancement in the art.

### BRIEF SUMMARY OF THE INVENTION

An illustrative composition according to the present invention comprises a mixture of (a) an anticoagulation polysaccharide covalently bonded to a hydrophobic agent, and (b) a solubilizer. Illustrative anticoagulation polysaccharides according to the present invention include unfractionated heparin, low molecular weight heparin, heparan sulfate, heparinoids, and mixtures or any two or more thereof. Illustrative hydrophobic agents according to the present invention include bile acids, sterols, alkanoic acids, and mixtures of any two or more thereof. Illustrative bile acids according to the present invention include cholic acid, deoxycholic acid, chenodeoxycholic acid, lithocholic acid, ursocholic acid, ursodoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, hyodeoxycholic acid, mixtures of any two or more thereof. Illustrative sterols according to the present invention include cholestanol, coprostanol, cholesterol, epicholesterol, ergosterol, ergocalciferol, and mixtures of any two or more thereof. Illustrative alkanoic acids according to the present invention include butyric acid, valeric acid, caproic acid, caprylic acid, capric acid, lauric acid, myristic acid, palmitic acid, stearic acid, and mixtures or any two or more thereof.

Illustrative solubilizers according to the present invention include polyethylene oxide, hydroxyalkyl cellulose, hydroxypropylalkyl cellulose, polyvinyl alcohol, polyvinylpyrrolidone, copovidone, sodium carboxymethyl cellulose, carbopol, sodium alginate, xanthan gum, locust bean gum, cellulose gum, gellan gum, tragacanth gum, karaya gum, guar gum, acacia gum, poloxamer, cyclodextrin, a dextrin derivative, a surfactant, and mixtures of any two or more thereof. Illustrative surfactants according to the present invention include anionic surfactants, non-anionic surfactants, zwitterionic surfactants, and mixtures of any two or more thereof.

Another illustrative embodiment of the present invention comprises a method of making an anticoagulation composition, the method comprising:

(a) dispersing or dissolving a solubilizer in a solvent, or heating the solubilizer, to achieve a liquid solubilizer, and then adsorbing the liquid solubilizer to an inert porous additive to result in adsorbed particles;

(b) mixing an anticoagulation polysaccharide covalently bonded to a hydrophobic agent with the adsorbed particles to result in a mixture; and

(c) granulating the mixture.

The granulated mixture can be pressed into a tablet or chewable tablet, or a capsule or a sachet can be filled with the granulated mixture. The granulated mixture can also be coated with a coating layer. Alternatively, the granulated mixture can be formed into a hydrophilic gel or syrup, or the granulated mixture can be dispersed in an aqueous or oil suspension.

Another illustrative embodiment of the present invention comprises a method of making an anticoagulation composition, the method comprising:

(a) mixing an anticoagulation polysaccharide covalently bonded to a hydrophobic agent with a solubilizer and a pharmaceutically acceptable additive to result in a mixture; and

(b) optionally, granulating the mixture by dry granulation, wet granulation, melt-granulation, fluidized granulation, high-speed rotation, direct compression, molding, or extrusion to result in a granulated mixture.

Still another illustrative embodiment of the invention comprises a method of making an anticoagulation composition, the method comprising:

(a) dispersing or dissolving an anticoagulation polysaccharide covalently bonded to a hydrophobic agent in a solubilizer to result in a solubilized anticoagulation polysaccharide;

(b) optionally, dispersing or dissolving the solubilized anticoagulation polysaccharide in a solvent, or heating the solubilized anticoagulation polysaccharide, to result in a solubilized anticoagulation polysaccharide liquid;

(c) adsorbing the solubilized anticoagulation polysaccharide or solubilized anticoagulation polysaccharide liquid to an inert porous additive to result in adsorbed particles; and

(d) granulating the adsorbed particles by dry granulation, wet granulation, melt-granulation, fluidized granulation, high-speed rotation, direct compression, molding, or extrusion with a pharmaceutically acceptable additive to result in a granulated mixture.

### BRIEF DESCRIPTION OF THE SEVERAL VIEWS OF THE DRAWINGS

FIG. 1 shows concentration profiles as measured by FXa assay of heparin or heparin-DOCA conjugate with or without a solubilizer after oral administration in rats; data are plotted as mean±SEM, n=4.

### DETAILED DESCRIPTION

Before the present compositions and methods are disclosed and described, it is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

The publications and other reference materials referred to herein to describe the background of the invention and to provide additional detail regarding its practice are hereby incorporated by reference. The references discussed herein are provided solely for their disclosure prior to the filing date of the present application. Nothing herein is to be construed as an admission that the inventors are not entitled to antedate such disclosure by virtue of prior invention.

It must be noted that, as used in this specification and the appended claims, the singular forms “a,” “an,” and “the” include plural referents unless the context clearly dictates otherwise. Thus, for example, reference to “an anticoagulation polysaccharide” includes reference to a mixture of two or more anticoagulation polysaccharides, reference to “a bile acid” includes reference to two or more of such bile acids, and reference to “the hydrophobic agent” includes reference to two or more of such hydrophobic agents.

Unless defined otherwise, all technical and scientific terms used herein have the same meanings as commonly understood by one of ordinary skill in the art to which this invention belongs.

In describing and claiming the present invention, the following terminology will be used in accordance with the definitions set out below.

As used herein, “comprising,” “including,” “containing,” “characterized by,” and grammatical equivalents thereof are inclusive or open-ended terms that do not exclude additional, unrecited elements or method steps. “Comprising” is to be interpreted as including the more restrictive terms “consisting of” and “consisting essentially of.” As used herein, “consisting of” and grammatical equivalents thereof exclude any element, step, or ingredient not specified in the claim. As used herein, “consisting essentially of” and grammatical equivalents thereof limit the scope of a claim to the specified materials or steps and those that do not materially affect the basic and novel characteristic or characteristics of the claimed invention.

As used herein, “anticoagulation polysaccharide” means, without limitation, unfractionated heparin, low molecular weight heparin, heparan sulfate, heparinoids, or mixtures of any two or more thereof.

As used herein, “hydrophobic agent” means, without limitation, a bile acid, a sterol, an alkanoic acid, or mixtures of any two or more thereof.

As used herein, “bile acid” means, without limitation, cholic acid, deoxycholic acid, chenodeoxycholic acid, lithocholic acid, ursocholic acid, ursodoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, hyodeoxycholic acid, or mixtures of any two or more thereof.

As used herein, “sterol” means, without limitation, cholestanol, coprostanol, cholesterol, epicholesterol, ergosterol, ergocalciferol, or mixtures of any two or more thereof.

As used herein, “alkanoic acid” means alkanoic acids of about 4 to about 20 carbon atoms. Illustrative alkanoic acids according to the present invention include butyric acid, valeric acid, caproic acid, caprylic acid, capric acid, lauric acid, myristic acid, palmitic acid, stearic acid, and mixtures of any two or more thereof.

An illustrative embodiment of the invention comprises a pharmaceutical composition containing an amphiphilic anticoagulation polysaccharide and a solubilizer, which solubilizer minimizes interference on the binding of the anticoagulation polysaccharide to the mucous layers and the bile acid transporters found in the GI tract. This interference on binding is due to the formation of self-assembled nanoparticles by hydrophobic moieties, such as bile acids, on the amphiphilic anticoagulation polysaccharide to which the hydrophobic moieties are conjugated. Reducing or eliminating the interference on binding in the GI tract enhances the solubility, absorption, and permeability of the drug after oral administration.

Ultimately, another illustrative embodiment of this invention provides convenience to patients by providing an oral anticoagulation formulation, which is an improvement over prior anticoagulation formulations that had been available only in injectable form.

Illustrative embodiments of this invention also relate to pharmaceutical compositions of anticoagulation polysaccharides, and a method of their manufacture, which pharmaceutical compositions are intended to increase the absorption rate by means of enhanced solubility of amphiphilic anticoagulation polysaccharides in the GI tract.

Illustrative amphiphilic anticoagulation polysaccharides, such as heparin-deoxycholic acid (heparin-DOCA), used as an effective ingredient in illustrative embodiments of the present invention are described in U.S. Pat. No. 6,245,753 and U.S. Pat. No. 6,656,922. For example, a heparin-bile acid derivative has been synthesized by conjugation of hydrophobic bile acid components to the negatively charged hydrophilic heparin molecule to enhance the absorption through the GI membrane. The conjugated bile components caused the adhesion to the bile acid transporters on the GI tract thereby increase concentration of heparin derivatives on the surface of GI membrane. Additionally, the conjugation of hydrophobic bile components on the heparin molecule induced the increase of partition coefficient through GI membrane and this made it more readily permeate and absorbed through membrane. However, the conjugated hydrophobic bile components such as deoxycholic acid induced the formation of self-assembled nanoparticles due to hydrophobic interactions in the GI tract before adhesion to the bile acid transporters and to the mucous layers. For this reason, absorption through the GI tract can be inhibited by prevention of adhesion of the conjugated bile components to the bile acid transporters and mucous layers.

Accordingly, the present invention reduces the formation of self-assembled nanoparticles and enhances adhesion of conjugated bile components to the bile acid transporters and mucous layers, thereby improving the oral absorption.

Illustrative examples of the solubilizer that may be used according to the present invention include polyethylene oxide, hydroxyalkyl cellulose, hydroxypropylalkyl cellulose, polyvinyl alcohol, polyvinylpyrrolidone, copovidone, sodium carboxymethyl cellulose, carbopol, sodium alginate, xanthan gum, locust bean gum, cellulose gum, gellan gum, tragacanth gum, karaya gum, guar gum, acacia gum, poloxamer, cyclodextrin, dextrin derivatives, surfactants, and mixtures thereof.

Illustrative examples of the surfactant that may be used according to the present invention include, but are not limited to, anionic surfactants, nonanionic surfactants, zwitterionic surfactants, and mixtures thereof. Typically, the surfactant comprises any one or more of alkyl- and alkylallyl-poly(oxyethylene)ethers, alkylallylformaldehyde condensed poly(oxyethylene)ethers, block copolymers that comprise poly(oxypropylene) as their lipophilic part, poly(oxyethylene)ethers of glycerin esters, poly(oxyethylene)ethers of sorbitan esters, poly(oxyethylene)sorbitan fatty acid esters, polyethylene glycol fatty acid esters, glycerin esters, sorbitan esters, propylene glycol esters, sugar esters, fatty acid alkanol amides, poly(oxyethylene) fatty acid amides, poly(oxyethylene)alkyl amines, polyglycolated glycerides, poly(oxyethylene) caster oils, poly(oxyethylene) hydrogenated caster oils, sorbitan fatty acid esters, monoglycerides of fatty acids, diglycerides of fatty acids, triglycerides of fatty acids, sugar fatty acid esters, bile salts, mixed micelles of bile salts and lecithin, glucose esters, and vitamin E TPGS (D-α-tocopheryl polyethylene glycol 1000 succinate).

Illustrative examples of the surfactant include, but are not limited to, Solutrol HS 15 (Macrogol-15-hydroxystearate); Cremophor RH 40 (Polyoxyl 40 Hydrogenated Castor Oil); Cremophor A6 (Macrogol(6) cetostearyl ether); Cremophor A25 (Macrogol(25) cetostearyl ether); Labrasol (Caprylocaproyl macrogolglycerides (Polyoxylglycerides)); Transcutol (Diethylene glycol monoethyl ether); Tweens (polysorbate 20, 21, 40, 61, 65, 80, 81, 85, 120); Poloxamers 124, 188, 237, 338, 407 (Polyoxyethylene polyoxypropylene block copolymer); Nikkol HCO-40 (polyoxyethylene glycolated natural or hydrogenated castor oil); Myrj 45 (polyoxyethylene(8) stearate); Tagat L (polyoxyethylene(30) mono-laurate); Marlosol 1820 (polyoxyethylene(20) stearate); Marlosol OL 15 (polyoxyethylene(15) oleate); Brjj 52 (polyoxyethylene(2) cetyl alcohol); Brjj 96 (polyoxyethylene(10) oleyl ether); Brjj 700 (polyoxyethylene(100) stearyl alcohol); Volpo 015 (polyoxyethylene(15) oleyl ether); Marlowet OA30 (polyoxyethylene(30) oleyl ether); Marlowet LMA 20 (polyoxyethylene(20) oleyl ether); Syperonic PE L44 (polyoxyethylene-polyoxypropylene copolymer); Syperonic F127 (polyoxyethylene-polyoxypropylene copolymer); Labrafil M1994CS (Oleoyl macrogolglycerides (polyoxylglycerides)); Labrafil M2125CS (Linoleoyl macrogolglycerides(polyoxylglycerides)); Labrafil M2130CS (Lauroyl macrogolglycerides(polyoxylglycerides)); Labrafac PG (Propylene glycol dicaprylocaprate); Imbitor (caprylic acid/capric acid mono- and di-glyceride); sorbitan mono-stearate; sorbitan tri-stearate; sorbitan mono-oleate; polyethylene glycol mono-oleate; Miglyol 840 (propylene glycol dicaprylate); Gelucire 44/14 (Lauroyl macrogolglycerides (polyoxylglycerides)); Gelucire 50/13 (Stearoyl macrogolglycerides(polyoxylglycerides)); Plurol oleique CC 497 (Polyglyceryl oleate); Lauroglycol FCC (Propylene Glycol Laurate); Capryol PGMC (Propylene glycol caprylate); Lauroglycol 90 (Propylene glycol monolaurate); Capryol 90 (Propylene glycol monocaprylate); fatty acid salts; α-sulfonyl fatty acid esters; linear alkyl benzene sulfonic acid salts; alkyl sulfonic acid ester salts; alkyl ether sulfonic acid ester salts; alpha olefin sulfonic acid salts; alkyl sulfonic acid salts; sodium lauryl sulfate; alkyl amino fatty acid salts; alky betaines; lecithins; lauryl di-methyl betaines; and mixtures thereof.

The above mentioned solubilizers have both hydrophobic and hydrophilic moieties. The hydrophobic portion of the heparin derivative binds to the hydrophobic moiety of the solubilizer, while the hydrophilic moiety of the solubilizer attracts water from the environment to prevent aggregation due to binding between hydrophobic constituents conjugated to heparin. As a result, the bile acid constituent conjugated to heparin is exposed and can easily contact the bile acid transporters and mucous layers in the GI tract, resulting in a concentration gradient on the GI membrane surface. Finally, due to this concentration gradient, amphiphilic heparin derivatives penetrate through the GI membrane and are absorbed.

Also, illustrative pharmaceutical composition according to the present invention may further comprise at least one pharmaceutically acceptable additive, e.g., binders, diluents, swelling agents, lubricants, antioxidants, coating agent, effervescent agents, and flavors.

Examples of the binder includes, but are not-limited to, polyvinylpyrrolidone, copovidone, gelatin, starch, sucrose, methyl cellulose, ethyl cellulose, hydroxyethyl cellulose, hydroxypropyl cellulose, hydroxypropyl alkyl cellulose, and mixtures thereof.

Examples of the diluent include, but are not-limited to, lactose, dextrin, mannitol, sorbitol, starch, microcrystalline cellulose, calcium hydrogen phosphate, anhydrous calcium hydrogen phosphate, calcium carbonate, sugars, colloidal silicon dioxide, calcium silicate, silicon dioxide, magnesium aluminosilicate, and mixtures thereof.

Examples of the swelling agent include, but are not-limited to, cross-linked polyvinylpyrrolidone, cross-linked sodium carboxymethyl cellulose, cross-linked calcium carboxymethyl cellulose, cross-linked carboxymethyl cellulose, sodium starch glycolate, carboxymethyl starch, sodium carboxymethyl starch, potassium methacrylate-divinylbenzene copolymer, amylose, cross-linked amylose, starch derivatives, microcrystalline cellulose and cellulose derivatives, cyclodextrin and dextrin derivatives, and mixtures thereof.

Also, the formulation of the present invention may further comprise a coating layer to provide color, stability, release control, initial burst release prevention, and taste masking of a drug.

Examples of the coating agent that may be used in such a coating process include ethylcellulose, shellac, ammonio methacrylate copolymer, methacrylic acid copolymer, aminoalkyl methacrylate copolymer, methacrylic copolymer, methacrylate copolymer, polyvinylacetate, polyvinylpyrrolidone, polyvinylalcohol, hydroxymethyl cellulose, hydroxyethyl cellulose, hydroxypropyl cellulose, hydroxybutyl cellulose, hydroxypentyl cellulose, hydroxypropyl methyl cellulose, hydroxypropyl butyl cellulose, hydroxypropyl pentyl cellulose, hydroxyalkyl cellulose phthalate, sodium cellulose acetate phthalate, cellulose acetyl phthalate, cellulose ether phthalate, an anionic copolymer of methacrylic acid and methyl or ethyl methacrylate, hydroxypropyl methyl cellulose phthalate, hydroxylpropyl methyl cellulose acetate succinate, cellulose acetyl phthalate, Acryl-EZE (Colorcon Co.), Opadry (Colorcon Co.), and mixtures thereof.

The coating layer may further comprise lubricants, plasticizers, as well as colorants, antioxidants, tale, titanium dioxide, flavors, or mixtures thereof. Examples of the plasticizer include castor oil, fatty acids, substituted triglyceride and glyceride, triethyl citrate, polyethylene glycol having a molecular weight from about 300 to about 50000 Da and their derivatives, and mixtures thereof.

Examples of the lubricant include, but are non-limited to, stearic acid, stearic acid salt, talc, corn starch, carnauba wax, light anhydrous silicic acid, magnesium silicate, titanium oxide, microcrystalline cellulose, Macrogol 4000 and 6000, isopropyl myristate, calcium hydrogen phosphate, and mixtures thereof.

Illustrative embodiments of the present invention may also comprise a method wherein the heparin derivative and/or solubilizer and/or a pharmaceutically acceptable additive can be prepared as granules or mixtures. The granules may be, but are not limited to, dried, wet, melted, or fluidized granules prepared by such processes as kneading, high-speed rotation, direct compression, molding, or extrusion, as are well known in the art. The granules may be prepared by, but are not limited to, dissolving or dispersing the heparin derivative and/or solubilizer in the solvent and then drying to enhance the solubilizing effect.

The granules may be prepared according to the following method, but are not limited to this method. For solubilizers in liquid or semi-solid states at room temperature, a solution made by dispersing or dissolving in solvent or by heating is adsorbed to the inert and porous additive. The adsorbed particles are then mixed with heparin derivatives and/or a pharmaceutically acceptable additive. The granules can be obtained by granulating the resulting mixture through dry-granulation, wet-granulation, melt-granulation, fluidized granulation, high-speed rotation, direct compression, molding, or extrusion process, according to methods well known in the art.

The granules or mixture can be further pressed into a tablet or a chewable tablet or filled into a capsule or a sachet. Alternatively, a part of the composition containing a heparin derivative and a part containing a solubilizer, or a part containing the heparin derivative and solubilizer and a part containing pharmaceutical excipients can be formulated separately or mixed into a tablet, a chewable tablet, a capsule, or a sachet.

The granules or mixture of the present invention may further be formulated into a hydrophilic gel, syrup, or a dispersion in an aqueous or oil suspension.

In other illustrative embodiments of the present invention, a formulation comprising a heparin derivative and/or a solubilizer and/or pharmaceutical additives can be prepared into a liquid, semi-solid, emulsion, micro-emulsion, or suspension form and then filled into a capsule or sachet.

The formulation of the present invention can increases the solubility and oral absorption of heparin derivatives; therefore, it can be expected to improve adaptation of taking medicine and patience compliance by enabling oral administration of heparin, which previously could only be administered intravenously or subcutaneously.

The present invention is further described and illustrated in Examples provided below, which are, however, not intended to limit the scope of the present invention.

### Examples

### Test Example 1

### Synthesis of Heparin-Deoxycholic Acid (Heparin-DOCA) Conjugates

Heparin-DOCA was synthesized as described in U.S. Pat. No. 6,656,922. In brief, DOCA was activated by reaction with N-hydroxylsuccinimide (HOSU) and dicyclohexylcarbodiimide (DCC). The activated DOCA was then reacted with ethylene diamine to form deoxycholylethylamine. Carboxyl functional groups of low molecular weight heparin (LMWH), where salts had been removed, were activated using EDCA and then coupled with the amine group of deoxycholylethylamine. The mole ratio between the LMWH and deoxycholylethylamine was 1:2. The resulting heparin-DOCA conjugate was then precipitated in ethanol, and the unreacted reagents were removed by centrifugation and ethanol washing three times. Ethanol was removed from the heparin-deoxycholic acid precipitate, then the precipitate was dissolved in distilled water and freeze dried, finally obtaining a white solid.

### Test Example 2

### Preparation of a Solution Containing Heparin-DOCA Conjugates and Solubilizer

To measure the formation of self-assembled nanoparticles in aqueous solutions of heparin-DOCA and to observe the prevention of particle formation due to a solubilizer, the solubilizers shown in Table 1 were added at a concentration of 0.1 mg/ml to 1 mg/ml of heparin-DOCA solution, and electrophoretic light scattering spectrophotometry (ELS-8,000) was used to measure the size of particles in the solution.

TABLE 1

| Materials | Particle Size (nm) |
|---|---|
| Heparin-DOCA | 369.5 |
| Heparin-DOCA + Poly(oxyethylene)(20) sorbitan | N.D. |
| mono-laurate | |
| Heparin-DOCA + Poly(oxyethylene)(80) sorbitan | N.D. |
| mono-olyate | |
| Heparin-DOCA + Poly(oxyethylene)(2) cetyl alcohol | N.D. |
| Heparin-DOCA + Poly(oxyethylene)(100) stearyl | N.D. |
| alcohol | |
| Heparin-DOCA + Polyoxyl 35 castor oil | N.D. |
| Heparin-DOCA + Poloxamer 188 | N.D. |
| Heparin-DOCA + Poloxamer 407 | N.D. |
| Heparin-DOCA + Sodium lauryl sulfate | N.D. |
| Heparin-DOCA + Povidone | N.D. |
| Heparin-DOCA + Caprylocaproyl macrogolglycerides | N.D. |
| Heparin-DOCA + Diethylene glycol monoethyl ether | 359.4 |

N.D. indicates that particles were not detected.

As shown in the results above, in the case of heparin-deoxycholic acid, nanoparticles of 370 nm were formed in aqueous solution due to hydrophobic interactions of hydrophobic deoxycholic acid moieties conjugated to the heparin molecule. However, except for diethylene glycol monoethyl ether, when all other solubilizers listed above were added, the heparin-DOCA conjugates were completely dissolved, because the aggregation phenomenon owing to the hydrophobic interaction of hydrophobic deoxycholic acid was prevented by the solubilizer. Therefore, in the presence of a solubilizer the hydrophobic portion of the heparin conjugate is exposed to the aqueous environment in the solution. In the GI tract, the solubilizer renders a bile acid moiety, for example, accessible for binding with the bile acid transporters. Thus, ready absorption through the GI membrane is expected.

### Comparative Examples 1 to 6 and Examples 1 to 10

### Preparation of a Solution Containing Heparin-DOCA Conjugates and Solubilizer

To measure the oral absorption of a heparin-deoxycholic acid formulation containing a solubilizer, materials listed in Table 2 were added to 400 μl of water and then mixed into a solution.

In the case of Comparative Example 3, a heparin-deoxycholic acid conjugate was dissolved in 10% dimethly sulfoxide (DMSO) solution, followed by dispersion using a probe-type ultrasound disperser, and followed by freeze-drying at −80° C. for 3 days to obtain heparin-deoxycholic acid conjugate powder containing dimethyl sulfoxide.

TABLE 2

| | Material | Amount (mg) |
|---|---|---|
| Comparative | LMWH | 2.5 |
| Example 1 | | |
| Comparative | Heparin-DOCA | 2.5 |
| Example 2 | | |
| Comparative | Heparin-DOCA + DMSO | 2.5 |
| Example 3 | | |
| Comparative | LMWH + Poloxamer 407 | 2.5 + 0.25 |
| Example 4 | | |
| Comparative | LMWH + Caprylocaproyl | 2.5 + 50 μl |
| Example 5 | macrogolglycerides | |
| Comparative | LMWH + sodium lauryl sulfate | 2.5 + 0.09 |
| Example 6 | | |
| Example 1 | Heparin-DOCA + Caprylocaproyl | 2.5 + 50 μl |
| | macrogolglycerides | |
| Example 2 | Heparin-DOCA + sodium lauryl sulfate | 2.5 + 0.09 |
| Example 3 | Heparin-DOCA + Linoleoyl | 2.5 + 0.25 |
| | macrogolglycerides | |
| Example 4 | Heparin-DOCA + Polyoxyl 35 castor oil | 2.5 + 0.25 |
| Example 5 | Heparin-DOCA + Poly(oxyethylene)(40) | 2.5 + 0.25 |
| | stearate | |
| Example 6 | Heparin-DOCA + Vitamin E TPGS (D-α- | 2.5 + 0.25 |
| | tocopheryl polyethylene glycol 1000 | |
| | succinate) | |
| Example 7 | Heparin-DOCA + Stearoyl | 2.5 + 0.25 |
| | macrogolglycerides | |
| Example 8 | Heparin-DOCA + Macrogol(25) | 2.5 + 0.25 |
| | cetostearyl ether | |
| Example 9 | Heparin-DOCA + Poloxamer 188 | 2.5 + 0.25 |
| Example 10 | Heparin-DOCA + Poloxamer 407 | 2.5 + 0.25 |

### Test Example 3

### In vivo Oral Absorption

To observe the effect of solubilizers on the oral absorption of heparin-deoxycholic acid conjugates as a result of increased its GI solubility and permeability, 10 mg/kg of heparin-deoxycholic acid conjugate solutions prepared according to the above examples were orally administered to rats. Blood samples were then collected and the platelets were separated from the samples to measure the blood concentration of heparin-deoxycholic acid conjugates.

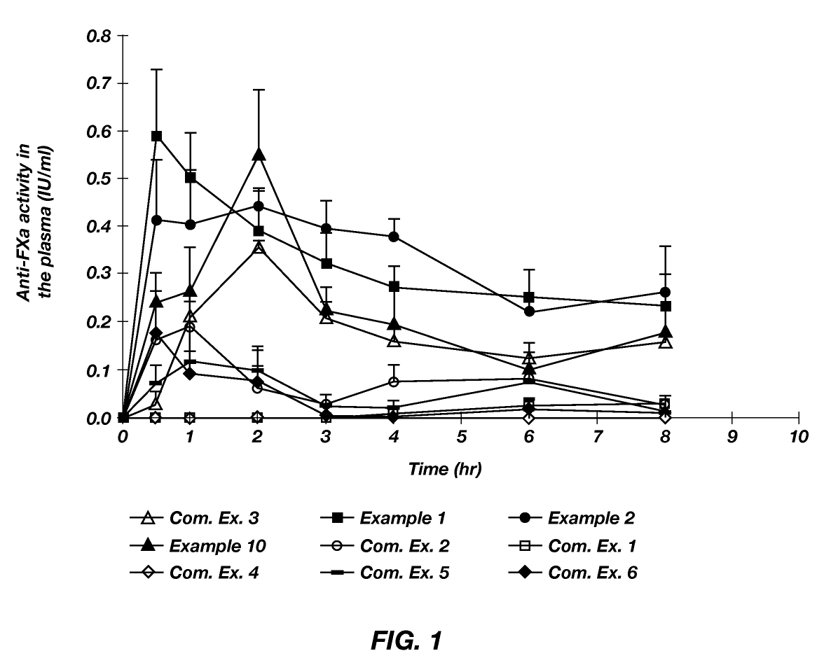

The concentration of heparin-DOCA conjugate in the plasma was determined by FXa assay, as shown in FIG. 1. From the time-concentration graph, the pharmacokinetic parameters of heparin-DOCA were calculated (Table 3).

TABLE 3

| | AUC (IU · hr/ml) | Cₘₐₓ(IU/ml) | Tₘₐₓ(hr) |
|---|---|---|---|
| Comp. Ex. 1 | N.D. | N.D. | N.D. |
| Comp. Ex. 2 | 0.592 ± 0.194 | 0.196 ± 0.058 | 1.0 ± 0.5 |
| Comp. Ex. 3 | 1.283 ± 0.142 | 0.297 ± 0.049 | 2.0 ± 1.0 |
| Comp. Ex. 4 | N.D. | N.D. | N.D. |
| Comp. Ex. 5 | 0.370 ± 0.106 | 0.176 ± 0.054 | 1.2 ± 0.4 |
| Comp. Ex. 6 | 0.281 ± 0.089 | 0.220 ± 0.041 | 0.7 ± 0.2 |
| Example 1 | 2.522 ± 0.205 | 0.693 ± 0.071 | 0.9 ± 0.4 |
| Example 2 | 2.336 ± 0.160 | 0.502 ± 0.075 | 2.3 ± 0.6 |
| Example 3 | 0.583 ± 0.206 | 0.212 ± 0.062 | 0.6 ± 0.1 |
| Example 4 | 0.983 ± 0.117 | 0.263 ± 0.038 | 1.3 ± 0.6 |
| Example 5 | 1.105 ± 0.369 | 0.259 ± 0.057 | 4.0 ± 1.8 |
| Example 6 | 1.128 ± 0.140 | 0.221 ± 0.019 | 2.3 ± 1.3 |
| Example 7 | 0.783 ± 0.010 | 0.188 ± 0.016 | 0.6 ± 0.1 |
| Example 8 | 0.510 ± 0.175 | 0.168 ± 0.012 | 3.6 ± 1.1 |
| Example 9 | 1.606 ± 0.173 | 0.398 ± 0.055 | 2.0 ± 0.7 |
| Example 10 | 1.463 ± 0.345 | 0.439 ± 0.106 | 1.0 ± 0.4 |

As shown in Table 3, heparin-DOCA (Example 2) was absorbed through the oral route compared to Comparative Example 2 (LMWH). The absorbed amount of heparin-DOCA in Examples 1, 2, 9, and 10 was higher than that in the comparative group (Comparative Example 2). Thus, the conjugated deoxycholic acid is readily exposed to the GI fluid by the action of solubilizer and easily adheres to the bile acid transporters thereof the oral absorption can be increased.

The oral absorption of Examples 1, 2, 9, and 10 was at least as effective as that of Comparative Example 3. Therefore, the solubilizer can improve the oral absorption of heparin-DOCA and replace an organic solvent such as DMSO.

### Examples 11 to 13

### Preparation of Matrix Tablet Comprising Heparin-DOCA

Heparin-DOCA, microcrystalline cellulose, poloxamer and/or sodium lauryl sulfate, lactose, and copovidone were passed through a No. 30 mesh screen and then mixed. Magnesium stearate was added to the resulting mixture and then mixed. Next, the resulting mixture was compressed to obtain a tablet. The tablet composition is shown in Table 4.

TABLE 4

| Ingredient | Example 11 | | Example 12 | | Example 13 | |
|---|---|---|---|---|---|---|
| Heparin-DOCA | 350 | mg | 350 | mg | 350 | mg |
| Microcrystalline cellulose | 80.5 | mg | 80.5 | mg | 80.5 | mg |
| Lactose | 80.5 | mg | 80.5 | mg | 80.5 | mg |
| Cross-linked sodium | 70 | mg | 70 | mg | 70 | mg |
| carboxymethyl cellulose | | | | | | |
| Copovidone | 42 | mg | 42 | mg | 42 | mg |
| Sodium lauryl sulfate | 70 | mg | — | | — | |
| Poloxamer 188 | — | | 70 | mg | — | |
| Poloxamer 407 | — | | — | | 70 | mg |
| Magnesium stearate | 7 | mg | 7 | mg | 7 | mg |
| Total weight | 700 | mg | 700 | mg | 700 | mg |

### Examples 14 to 16

### Preparation of Coated Matrix Tablet Comprising Heparin-DOCA

The tablets prepared according to in Examples 11 to 19 were successively spray-coated with a mixture of hydroxypropyl methylcellulose phthalate or Acryl-EZE (Colorcon Co., West Point, Pa.) which is an enteric coating agent containing a colorant, in amounts shown in Table 5.

TABLE 5

| | Example 14 | Example 15 | Example 16 |
|---|---|---|---|
| Uncoated matrix tablet | Example 11 | Example 12 | Example 13 |
| Acryl-EZE | 70 | — | — |
| Hydroxypropyl | — | — | 35 |
| methylcellulose phthalate | | | |
| Opadry | — | 35 | — |
| Water | 630 | 548 | — |
| Ethanol* | — | — | 274 |
| Methylene chloride | — | — | 274 |

### Examples 17 and 18

### Preparation of Granules and Matrix Tablets Comprising Heparin-DOCA

Lactose and either colloidal silicon dioxide or calcium silicate were mixed in a high-speed mixer. To this mixture a dispersion or solution of heparin-DOCA and caprylocaproyl macrogolglycerides was slowly added and absorbed to the mixture with rotation. Then, the resulting granules were mixed with microcrystalline cellulose, copovidone, and cross-linked sodium carboxymethyl cellulose, to which magnesium stearate was added. The resulting mixture was compressed to obtain a tablet or filled into a capsule.

TABLE 6

| | Example 17 | | Example 18 | |
|---|---|---|---|---|
| Heparin-DOCA | 350 | mg | 350 | mg |
| Caprylocaproyl macrogol glycerides | 70 | mg | 70 | mg |
| Lactose | 30 | mg | 30 | mg |
| Colloidal silicon dioxide | 101 | mg | — | |
| Calcium silicate | — | | 101 | mg |
| Microcrystalline cellulose | 30 | mg | 30 | mg |
| Copovidone | 42 | mg | 42 | mg |
| Cross-linked sodium carboxymethyl | 70 | mg | 70 | mg |
| cellulose | | | | |
| Magnesium stearate | 7 | mg | 7 | mg |
| Total weight | 700 | mg | 700 | mg |

### Examples 19 and 20

### Preparation of Granules and Matrix Tablets Comprising Heparin-DOCA

Lactose, microcrystalline cellulose, cross-linked sodium carboxymethyl cellulose, and either colloidal silicon dioxide or calcium silicate were mixed in a high-speed mixer, and a solution prepared by heating the mixture of heparin-DOCA, linoleoyl macrogolglycerides, and povidone at 50° C. was slowly added and granulated. The resulting mixture was passed through No. 14 mesh screen to obtain granules. The granules were dried and passed through No. 16 mesh screen, to which a magnesium stearate was added. Then, the resulting mixture was compressed to obtain a tablet or filled into a capsule. The final composition of a tablet or a capsule was shown in Table 7.

TABLE 7

| | Example 19 | | Example 20 | |
|---|---|---|---|---|
| Heparin-DOCA | 350 | mg | 350 | mg |
| Linoleoyl macrogol glycerides | 70 | mg | 70 | mg |
| Lactose | 30 | mg | 30 | mg |
| Colloidal silicon dioxide | 101 | mg | — | |
| Calcium silicate | — | | 101 | mg |
| Microcrystalline cellulose | 30 | mg | 30 | mg |
| Povidone | 42 | mg | 42 | mg |
| Cross-linked sodium carboxymethyl | 70 | mg | 70 | mg |
| cellulose | | | | |
| Magnesium stearate | 7 | mg | 7 | mg |
| Total weight | 700 | mg | 700 | mg |

### Examples 21 and 22

### Preparation of Granules and Coated Matrix Tablets Comprising Heparin-DOCA

Heparin-DOCA and polyvinylpyrrolidone with or without Labrasol (caprylocaproyl macrogolglycerides(polyoxylglycerides)) were dissolved in water. At the same time, lactose, microcrystalline cellulose, and cross-linked sodium carboxymethyl cellulose were put into the cylindrical column of the fluidized bed and then fluidized by blowing. When blowing became stabilized, the prepared solution was sprayed continuously. When the solution was sprayed onto the blowing excipients, it bound the excipients together and then formed granules, and thereby the drug was shielded by the other excipients and yielding good compressibility. The wetted granules were further dried by blowing in the bed simultaneously. The granules were dried and passed through a no. 16 mesh screen, to which Poloxamer 407 was added. Finally, the mixture was lubricated by adding magnesium stearate and magnesium aluminum metasilicate. The resulting mixture was compressed to obtain a tablet and then coated with Opadry White (85F18422). The final composition of coated tablets are shown in Table 8.

TABLE 8

| | |
|---|---|
| Example 21 | Example 22 |

| Heparin-DOCA | 100 | mg | 100 | mg |
|---|---|---|---|---|
| Polyvinylpyrrolidone | 6 | mg | 6 | mg |
| (Kollidon K30) | | | | |
| Labrasol | — | | 6 | mg |
| Microcrystalline cellulose | 24 | mg | 24 | mg |
| Lactose | 24 | mg | 24 | mg |
| Cross-linked sodium | 20 | mg | 20 | mg |
| carboxymethyl cellulose | | | | |
| Poloxamer 407 (Lutrol F 127, | 20 | mg | 20 | mg |
| micronized) | | | | |
| Magnesium stearate | 2 | mg | 2 | mg |
| Magnesium aluminum | 4 | mg | 4 | mg |
| metasilicate | | | | |
| Opadry White (85F18422) | 8 | mg | 8 | mg |
| Total weight | 208 | mg | 214 | mg |

The subject matter claimed is:

1. A composition comprising a mixture of (a) an anticoagulation polysaccharide covalently bonded to a hydrophobic agent, and (b) a solubilizer.

2. The composition of claim 1 wherein the anticoagulation polysaccharide comprises unfractionated heparin, low molecular weight heparin, heparan sulfate, heparinoids, or mixtures of any two or more thereof.

3. The composition of claim 1 wherein the hydrophobic agent comprises a bile acid, a sterol, an alkanoic acid, or a mixture of any two or more thereof.

4. The composition of claim 1 wherein the hydrophobic agent comprises a sterol.

5. The composition of claim 4 wherein the sterol comprises cholestanol, coprostanol, cholesterol, epicholesterol, ergosterol, ergocalciferol, or a mixture of any two or more thereof.

6. The composition of claim 1 wherein the hydrophobic agent comprises a bile acid.

7. The composition of claim 6 wherein the bile acid comprises cholic acid, deoxycholic acid, chenodeoxycholic acid, lithocholic acid, ursocholic acid, ursodoxycholic acid, isoursodeoxycholic acid, lagodeoxycholic acid, glycocholic acid, taurocholic acid, glycodeoxycholic acid, glycochenodeoxycholic acid, dehydrocholic acid, hyocholic acid, hyodeoxycholic acid, or a mixture of any two or more thereof.

8. The composition of claim 1 wherein the hydrophobic agent comprises an alkanoic acid of about 4 to about 20 carbon atoms.

9. The composition of claim 8 wherein the alkanoic acid comprises butyric acid, valeric acid, caproic acid, caprylic acid, capric acid, lauric acid, myristic acid, palmitic acid, stearic acid, or a mixture of any two or more thereof.

10. The composition of claim 1 wherein the solubilizer comprises polyethylene oxide, hydroxyalkyl cellulose, hydroxypropylalkyl cellulose, polyvinyl alcohol, polyvinylpyrrolidone, copovidone, sodium carboxymethyl cellulose, carbopol, sodium alginate, xanthan gum, locust bean gum, cellulose gum, gellan gum, tragacanth gum, karaya gum, guar gum, acacia gum, poloxamer, cyclodextrin, a dextrin derivative, a surfactant, or a mixture of any two or more thereof.

11. The composition of claim 10 wherein the surfactant is present and comprises an anionic surfactant, a non-anionic surfactant, a zwitterionic surfactant, or a mixture of any two or more thereof.

12. The composition of claim 1, further comprising one or more binder, diluent, swelling agent, lubricant, antioxidant, coating agent, effervescent agent, flavor, or mixtures thereof.

13. The composition of claim 1 further comprising a coating layer encapsulating the mixture.

14. A method of making an anticoagulation composition, the method comprising:
(a) dispersing or dissolving a solubilizer in a solvent or heating the solubilizer, to achieve a liquid solubilizer, and then adsorbing the liquid solubilizer to an inert porous additive to result in adsorbed particles;
(b) mixing an anticoagulation polysaccharide covalently bonded to a hydrophobic agent with the adsorbed particles to result in a mixture; and
(c) granulating the mixture.

15. The method of claim 14, further comprising pressing the granulated mixture into a tablet or chewable tablet, or filling a capsule or a sachet with the granulated mixture.

16. The method of claim 15, further comprising coating the tablet, chewable tablet, capsule, or sachet with a coating layer.

17. The method of claim 14, further comprising coating the granulated mixture with a coating layer.

18. The method of claim 14, further comprising formulating the granulated mixture into a hydrophilic gel or syrup, or dispersing the granulated mixture in an aqueous or oil suspension.

19. A method of making an anticoagulation composition, the method comprising:
(a) mixing an anticoagulation polysaccharide covalently bonded to a hydrophobic agent with a solubilizer and a pharmaceutically acceptable additive to result in a mixture; and
(b) optionally, granulating the mixture by dry granulation, wet granulation, melt-granulation, fluidized granulation, high-speed rotation, direct compression, molding, or extrusion to result in a granulated mixture.

20. The method of claim 19, further comprising pressing the granulated mixture into a tablet or chewable tablet, or filling a capsule or a sachet with the granulated mixture.

21. The method of claim 20, further comprising coating the tablet, chewable tablet, capsule, or sachet with a coating layer.

22. The method of claim 19, further comprising coating the granulated mixture with a coating layer.

23. A method of making an anticoagulation composition, the method comprising:
(a) dispersing or dissolving an anticoagulation polysaccharide covalently bonded to a hydrophobic agent in a solubilizer to result in a solubilized anticoagulation polysaccharide;
(b) optionally, dispersing or dissolving the solubilized anticoagulation polysaccharide in a solvent, or heating the solubilized anticoagulation polysaccharide, to result in a solubilized anticoagulation polysaccharide liquid;
(c) adsorbing the solubilized anticoagulation polysaccharide or solubilized anticoagulation polysaccharide liquid to an inert porous additive to result in adsorbed particles; and
(d) granulating the adsorbed particles by dry granulation, wet granulation, melt-granulation, fluidized granulation, high-speed rotation, direct compression, molding, or extrusion with a pharmaceutically acceptable additive to result in a granulated mixture.

24. The method of claim 23, further comprising pressing the granulated mixture into a tablet or chewable tablet, or filling a capsule or a sachet with the granulated mixture.

25. The method of claim 24, further comprising coating the tablet, chewable tablet, capsule, or sachet with a coating layer.

26. The method of claim 23, further comprising coating the granulated mixture with a coating layer.

27. A method of making an anticoagulation compound, the method comprising:
(a) dispersing or dissolving an anticoagulation polysaccharide covalently bonded to a hydrophobic agent, binder, and, optionally, a solubilizer, in solvent to result in a solubilized anticoagulation polysaccharide;
(b) optionally, dispersing or dissolving the solubilized anticoagulation polysaccharide in a solvent, or heating the solubilized anticoagulation polysaccharide, to result in a solubilized anticoagulation polysaccharide liquid;
(c) granulating the solubilized anticoagulation polysaccharide solution with a pharmaceutically acceptable additive to result in a granulated mixture, by wet granulation, melt granulation, fluidized granulation, high-speed rotation, molding, or extrusion; and
(d) adding solubilizer and/or a pharmaceutically acceptable additive to the granulated mixture.

28. The method of claim 27, further comprising pressing the granulated mixture into a tablet or chewable tablet, or filling a capsule or a sachet with the granulated mixture.

29. The method of claim 28, further comprising coating the tablet, chewable table, capsule, or sachet with a coating layer.

30. The method of claim 27, further comprising coating the granulated mixture with a coating layer.
